(19) United States
(12) Patent Application Publication
(10) Pub. No.: US 2026/0198645 A1
(43) Pub. Date: Jul. 16, 2026

(54) Forefoot Sulcus Device, and Orthotic and Footwear Using Same

(71) Applicant: Ian Kenton Engelman, Portland, ME (US)

(72) Inventor: Ian Kenton Engelman, Portland, ME (US)

(21) Appl. No.: 19/448,181

(22) Filed: Jan. 14, 2026

Related U.S. Application Data
(60) Provisional application No. 63/746,141, filed on Jan. 16, 2025.

(51) Int. Cl. A43B 7/1425; A61F 5/01

(52) U.S. Cl. CPC A43B 7/1425; A61F 5/0111

(57) ABSTRACT

A weight and/or force distribution sulcus device for use as a part of a foot orthotic or a shoe, as an independent sulcus section for attachment to a foot orthotic or a shoe, or incorporated in a shoe. The device constructed to operationally underly a foot in the plantar sulcus area and comprise a sulcus support bar which is adjustable at least in front to rear direction relative to the shoe or orthotic. A locking mechanism is provided to selectively lock the sulcus section and the orthotic body or shoe at a selectively locked distance therebetween. Sulcus sections befitting usage with an orthotic body or a shoe are also disclosed as separate and discrete embodiments.

### Related Applications

This application claims priority to U.S. Provisional Application 63/746,141 filed Jan. 16, 2025 to the present inventor. The 63/746,141 application is incorporated herein by reference in its entirety. Further, this application incorporates by reference U.S. Pat. No. 11,758,971, which was issued to the present inventor on Sep. 19, 2023, to the extent that this disclosure does not contradict the instant disclosure.

### FIELD OF THE INVENTION

The present invention relates generally to a device for distributing force and weight, and more specifically to a device which is operationally disposed under or near the plantar sulcus region of a foot, and beneficially modifies weight distribution and/or force transfer from the foot to the ground.

### BACKGROUND OF THE INVENTION

Foot orthotics operationally redistribute the weight of the body on the plantar surface of the foot. For increased comfort and to alleviate certain medical conditions such as metatarsalgia, the goal is to unweight the metatarsal heads or the ball of the foot. Traditionally, the method of unweighting the metatarsal heads is through a metatarsal pad. The metatarsal pad is an element of a foot orthotic that is positioned behind the metatarsal heads, but in some cases may constitute the orthotic itself.

A sulcus bar is a structural element operationally lying under the foot in the plantar sulcus region which is in front of the metatarsal heads and behind the proximal end of the toes, and extends upwardly in height, into the plantar sulcus cavity. An intermetatarsal bar is another, less common method of unweighing the metatarsal heads. The front to back position of the sulcus bar is important in the unweighting effect to the metatarsal heads. A small difference in the front to back position can make a significant difference in the sulcus bar efficacy and comfort.

U.S. Pat. No. 11,758,971 to the present inventor describes an orthotic having sulcus support. This orthotic allows setting of the back to front location and shape of the sulcus bar, however this location and shape are set during manufacture.

For brevity, in this disclosure, certain terms are considered equivalent. The terms “sulcus” and “plantar sulcus” are considered equivalent. The terms “sulcus bar” and “sulcus support bar” are considered equivalent. The terms “force distribution device”, “force and weight distribution device”, “weight distribution device, and “sulcus device” are considered equivalent. The term “intermetatarsal” may be abbreviated as “IM”, and may be used as location or disposition indicator, and/or in combination with structure such as an IM bar, which is equivalent to an intermetatarsal bar. An intermetatarsal bar generally refers to a bar which operationally lies underneath the foot between the first and second metatarsal heads. The IM bar may act as a connector bar connecting the orthotic and the sulcus bar.

In the present state of art, predicting the best front to back position of the sulcus bar during the fabrication of foot orthotics is difficult. Part of difficulty stems from differences in shoe designs. Some shapes of the upper part of shoes will bias the foot further forward relative the resting position of the foot orthotic at the bottom of the shoe. In such circumstances, the user will experience the sulcus bar as being set excessively towards the back, causing discomfort under the ball of the foot.

Another challenge to the sulcus bar efficacy stems from the differences between individuals, specifically from different size and quality of the cushioning fat pad under the ball of the foot. For some patients the fat pad is thicker and wider, and for others, it can be thinner and narrower. Therefore, a factor effecting the sulcus device efficacy is the shape and size of the sulcus and/or the IM bar. The most comfortable height and width of the bars in specific areas of the device is likely to vary from one individual to another.

A sulcus bar, may increase comfort and/or assist in correcting orthopedic impairments such as by unweighting the metatarsal heads. A sulcus device, implies at least a combination of a sulcus bar and an IM bar coupled to the sulcus barat least the sulcus bar and the IM bar is operationally disposed in the plantar sulcus region of a foot.

A sulcus device is a device which may be embodied in a shoe, an orthotic or a portion thereof, and/or in a sulcus section of an orthotic which may be manufactured separately for interaction with a shoe or an orthotic.

The foot and the sulcus device relative positioning is also a meaningful consideration. Small differences in tissue consistency of the foot can result in a variety of positional relationships between the skeletal foot and various parts of the sulcus device or portions thereof, especially in the front to back positioning, but also in side-to-side positioning. For instance, a stiffer thicker heel pad anterior to the calcaneus will push the foot back when the orthotic longitudinal arch presses into that heel pad. In this example, the result might be a positional misalignment of the sulcus bar relative to the metatarsal heads where the sulcus bar is disposed too far forward. In contrast, a foot with leaner tissue structure at the same anterior calcaneal area might result in a shift forward of the skeleton on the foot orthotic while weight bearing. In this case, the sulcus bar may be disposed too posterior to the metatarsal heads, and the user may experience pain from pressure on or near the metatarsal heads.

It is thus seen that a fixed position of the sulcus bar to the body of the foot orthotic leads to higher percentage of failures in unweighting the ball of the foot, resulting in patient discomfort or a failure of the therapeutic purpose of the orthotic. Furthermore, as foot orthotics are manufactures as a unit, and are oftentimes a special-made article which is made away from the healthcare provider office, changes to the placement of the sulcus bar relative to the orthotic are difficult and time consuming, while the patient discomfort continues. Furthermore, making a plurality of orthotics with differing prescribed positions is costly.

Thus, an heretofore unmet need is recognized: to allow adjustability of the sulcus support bar and/or other portions a sulcus device relative to the foot. Such adjustability will allow increased efficacy of the foot orthotic.

### SUMMARY

It is therefore an object of this disclosure to provide a solution to the need for adjustability of a sulcus bar relative to a foot orthotic or shoe.

To those goals, in an aspect of the invention there is provided a force and/or weight distribution device, equivalently known as a sulcus device. Embodiments of the sulcus device may be built into a shoe inner sole, incorporated fixedly or adjustably in a foot orthotic, or be embodied as a separate sulcus section to be attached to a footwear or an orthotic. The sulcus device comprises a sulcus bar and an intermetatarsal bar, the sulcus bar may be generally divided to a medial portion and/or a lateral portion. A sulcus device embodied as a sulcus section further comprises a stem for attachment to the footwear or the orthotic. For brevity, numerous examples provided in this specification will demonstrate principles utilizing a foot orthotic having the sulcus device adjustably attached thereto, and/or a sulcus section attachable to such orthotic or shoe, however, those aspects will be transferable to any footwear or stated differently, embodied in a shoe.

Thus, an aspect of the invention provides a foot orthotic or a shoe having an inner sole the orthotic comprising a body section having a frontal region, a sulcus section at least partially disposed forwardly to the frontal region, the sulcus section comprising an elongated intermetatarsal bar and a sulcus support bar coupled thereto, the sulcus support bar comprising at least one pad disposed forwardly of the frontal region and at an adjustable distance therefrom, the distance being adjustable at least in a front to rear direction, the elongated intermetatarsal bar coupling between to the body section and the sulcus support bar; and a locking mechanism adapted to selectively lock the sulcus support bar and the body section at the adjustable distance therebetween. In embodiments wherein the sulcus section is embodied in a shoe, the frontal region may be considered to be disposed about the forward third of the shoe.

The intermetatarsal bar may be formed integrally with, or be coupled to, the sulcus support bar.

In some embodiments the sulcus support bar has a rearwardly extending stem, at least a portion of the stem is dimensioned to be receivable within an elongated cavity defined by the body section of the orthotic, and be adjustably located therein and selectively locked at a distance relative thereto by the locking mechanism.

In some embodiments the locking mechanism comprises a screw locking the stem relative to a portion of the body section, or hook and loop fastening, wherein a first portion of the hook and loop fastening is coupled to the shoe or the orthotic, and the second portion of the hook and loop fastening is coupled to the sulcus device, such as to the stem.

In some embodiments the body or the inner sole defines a plurality of substantially parallel elongated cavities, each cavity being dimensioned for receiving the stem or a portion thereof therein, the cavities being disposed along the lateral axis of the shoe or the orthotic.

In some embodiments the pad has a lateral portion forming a backwardly extending arch, and in some embodiments the lateral portion pad extends to the sulcus cavity lying in front of the fourth, or in front of the fifth metatarsal head of a foot being supported by the orthotic or footwear.

In some embodiments, the sulcus bar pad comprises a medial portion disposed on opposing side of the intermetatarsal bar axis from the lateral portion, the lateral portion of the pad or a portion thereof being higher than an average height of the medial portion of the pad. It is noted however that the sulcus device, whether embodied in a shoe, orthotic or a discrete sulcus section, may comprise only a lateral pad portion, only a medial pad portion or both a lateral and a medial pad portions. The lateral and medial pad portions may be separate and independent pads or each portion may be formed as a portion of a single pad. A sulcus bar is formed integrally with, or coupled to, the intermetatarsal bar forwardly of the orthotic body or in the footwear toe region forwardly of the intermetatarsal bar rear end. The sulcus bar comprises at least one pad. It is noted that while this disclosure relates to a sulcus bar pad, a plurality of pads may be utilized, and in such embodiments, they are related to as various portions of the pad.

In an optional embodiment, the elongated intermetatarsal bar is formed integrally with the body and extending forwardly therefrom, and wherein the sulcus bar being adjustably coupled and lockable to the intermetatarsal bar.

As described, the sulcus device may be embodied in a discrete, independent sulcus section which may be provided for interaction with an orthotic or footwear. Thus, in an aspect of the invention, there is provided a sulcus section adapted for use in a foot orthotic having a body section, or in a shoe having an inner sole, the inner sole or the orthotic having an elongated cavity formed therein, the shoe or orthotic having a first part of a locking mechanism, the sulcus section comprising an elongated intermetatarsal bar having a longitudinal axis; a stem coupled to or integral with the intermetatarsal bar, the stem being dimensioned to be at least partially received within the cavity; and a sulcus support bar formed integrally to, or coupled to, the intermetatarsal bar. The stem forms a second part of the locking mechanism, which when cooperating with the first part of the locking mechanism, locks the stem at an adjustable position thereby locking the distance between the sulcus bar and the front end of the cavity, i.e. the end closer to the sulcus bar.

As described for the orthotic and/or the shoe above, the sulcus support bar may comprise a medial pad, a lateral pad, or a combination thereof, where the medial pad and the lateral extend on opposing sides of the longitudinal axis of the intermetatarsal bar. Optionally the lateral pad portion of the pad or a portion thereof forming a backwardly extending arch which may extend to the sulcus cavity in front of any of the second, third, fourth or fifth metatarsal. Optionally, the lateral pad portion further comprises a lateral height zone having a greater height than the average height of the medial pad portion.

Optionally, the stem comprises at least a portion having a plurality of teeth formed in at least one side thereof, the teeth are dimensioned to engage at least one protrusion in the cavity of the body or inner sole. Thus, the protrusion forms the first portion of the locking mechanism and the teeth form the second portion thereof.

In another aspect of the invention there is provided a shoe having an inner sole and constructed to cooperate with a sulcus device as described above, the inner sole having at least one elongated cavity formed therein the cavity being dimensioned to receive at least portion of the stem therein, the inner sole incorporating the first portion of a locking mechanism. Optionally, the shoe inner sole comprises a plurality of such elongated cavities, each dimensioned to receive at least portion of the stem therein, the cavities being substantially parallel to each other and extending along a lateral axis of the shoe.

The size of various portions of the sulcus device may be customized for individual's anatomical needs. By way of example intermetatarsal bar height and width increases to one individual would offer improved skeletal lock, or improved pressure distribution, while for another individual those increased dimensions may not be tolerated and a shorter and/or thinner stem design would offer a better fit. Similarly, size variations of the sulcus bar, both medially and laterally, may be provided to better fit individual needs. Thus, in some embodiments the sulcus device may be customized for individual user anatomical needs.

In some embodiments, the stem has a substantially trapezoidal or asymmetric cross-section, and in some embodiments the cavity has a substantially trapezoidal or asymmetric cross-section. Identity between the cross-section form of the stem and the cavity is not mandated.

A foot orthotic or a shoe inner sole may be constructed with rigid and/or semi-rigid material which provides the foundational support. It is common to attach a foam and/or cloth top cover over the orthosis body to provide some cushioning and continuity where it interfaces with the foot or socked foot.

The interface between the anatomical foot and a foot orthotic is sometimes hard to predict. The type of shoe might shift the foot either more medial or lateral depending on how tight or narrow the shoe is. The exact position of the foot orthotic within the well of the shoe might shift the foot orthotic either medial or lateral depending on the shape of the well and shape of the orthotic. Similarly, a foot may lie differently within a shoe or the shoe location for the cavity for the stem may not fit each individual foot. Therefore, for increased comfort of the user, it is desired to adjust the side-to-side position of the sulcus section and with it the sulcus bar, to better position it between the desired metatarsal heads. This can be achieved utilizing a plurality of cavities disposed side by side along the lateral axis, in the orthotic or the footwear, and the user or practitioner can reposition the support bar in either the medial or the lateral direction. However, the beginning and end points and/or length of the cavities does not have to be identical.

It is noted that the body of an orthotic functions by interacting with the underside surface of the foot. It is also noted that the body of the orthotic may only engage with part of the underside of the foot, meaning that its size might or might not include a heel, might or may not include the midfoot, might or may not include the fifth ray and the like, and even a metatarsal pad disposed operationally under one or more metatarsal heads is considered an orthotic. In some embodiments, the body of the orthotic could be constructed to be integral with an ankle foot orthosis, knee ankle foot orthosis, and the like.

Various aspects and components of the invention may be combined. Thus, by way of example, various sulcus bar pads and embodiments may be combined with various types of stems and locking mechanisms, the stem of any embodiment may have trapezoidal cross-section in any of the embodiments, the stem may interact with an orthotic or other footwear, and the like.

### BRIEF DESCRIPTION OF THE DRAWINGS

The summary above, and the following detailed description will be better understood in view of the enclosed drawings which depict details of preferred embodiments. It should however be noted that the invention is not limited to the precise arrangement shown in the drawings and that the drawings are provided merely as examples.

FIG. 1 depicts schematically a bottom view of a right foot orthotic embodying an aspect of the invention.

FIG. 2 depicts schematically a simplified top view of an embodiment for a right foot where the forefoot portion allowing lateral adjustability of the sulcus section.

FIG. 3 depicts schematically a top view of an embodiment for a left foot showing some optional features.

FIG. 4 depicts schematically a top view of a left foot orthotic embodiment utilizing peg and holes as a locking mechanism.

FIG. 5 depicts schematically an oblique bottom view showing a separated orthotic body section and sulcus section, utilizing peg and holes locking mechanism.

FIG. 6 depicts schematically an oblique bottom view of a right foot orthotic embodiment having a broad, asymmetrical stem.

FIG. 7 depicts schematically a top view of a left foot orthotic embodiment where the locking mechanism comprise cooperating sulcus bar and support bar.

FIG. 7A schematically depicts an exemplary sulcus bar of the embodiment of FIG. 6.

FIG. 8 depicts schematically a left foot interfacing with the foot orthotic embodying an aspect of the invention.

FIG. 9 depicts schematically an isometric view another embodiment of a forefoot weight and force distribution sulcus device, and FIG. 9A depicts schematically a rear view of the embodiment of FIG. 9.

FIG. 10 depicts schematically a frontal end view of an optional embodiment showing an orthotic offering lateral adjustment.

FIG. 11 depicts one more embodiment of the locking mechanism.

FIG. 12 depicts yet another embodiment of the locking mechanism.

FIG. 13 depicts yet another embodiment of the orthotic having replaceable forefoot section.

FIG. 14 depicts another embodiment of a sulcus device embodied within the shoe.

### DETAILED DESCRIPTION

The ensuing description, together with the accompanying figures, makes apparent to a person having ordinary skill in the pertinent art how the teachings of the disclosure may be practiced, by way of non-limiting examples. The figures are for the purpose of illustrative discussion and no attempt is made to show structural details of an embodiment in more detail than is necessary for a fundamental understanding of the disclosure. For the sake of clarity and simplicity, some objects depicted in the figures may not be drawn to scale.

While the figures sometimes depict a left or right foot or foot orthotic or shoe, it will be clear that corresponding contralateral side are merely mirror images of the depicted figures, and the invention scope extends equally to right and left foot and foot orthotic.

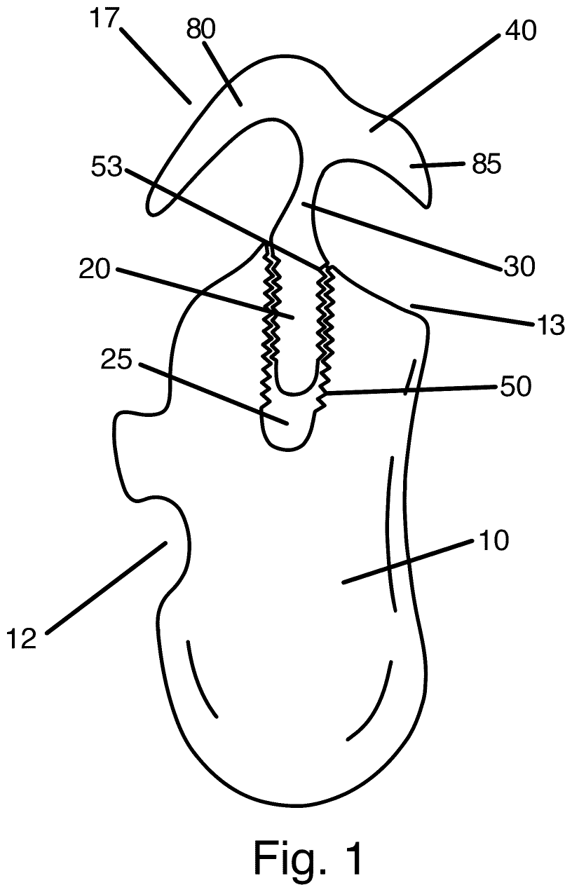

FIG. 1 depicts a bottom view of a right foot orthotic 10. The orthotic has a body section 12 having a heel region and a frontal region 13, and a sulcus section 17 comprising a stem 20, an intermetatarsal bar 30 and a sulcus bar 40. The sulcus bar comprises a pad, which in this embodiment extends both laterally 80 and medially 85. As was noted before the pad may comprise a plurality of pads, such as, by way of example, a separate lateral pad portion 80 and a separate medial pad portion 85, however both are considered equivalent to a single pad. The body section 12 of the orthotic defines a cavity 25 dimensioned to receive therein at least a portion of the stem 20. The cavity is generally shaped to cooperate with the stem but allow changing the relative location thereof, at least along the fore-aft direction. In the depicted embodiment, a plurality of periodically spaced teeth 53 are formed in the stem, and matching and opposing teeth 50 are formed by the body section and extend into the cavity 25. Generally, the teeth on the stem and in the cavity are periodic, however periodicity is not mandated and the number of teeth between the stem teeth 53 and the cavity teeth 50 does not have to equal. In some embodiments only a single tooth may be utilized on one of the cooperating parts stem and cavity, rather than a plurality as depicted by the example of FIG. 1. In some embodiments the period between two teeth is about 1 mm, or about 2 mm, or about 3 mm, or about 4 mm, or about 5 mm, however smaller and larger periods are acceptable. Similar spacing between any kind of protrusions and indentations may be adopted. Adjustment of the relative position of the sulcus section 17 and the body section 12 is done by positioning the stem teeth 53 to cooperate with the cavity teeth 50 at varying locations along the cavity. The stem can be separated from the body section of the orthotic, repositioned either forwards or backwards, and be attached again to the body section such that certain stem teeth 53 engage certain teeth in the cavity. By their nature, teeth form both protrusions and indentations. The mashing of the teeth of the two cooperating sections provide the desired adjustability and lockability in the desired spatial relationship. Alternatively, the sulcus section 17 can be removed altogether, which provide yet another form of adjustability, as the sulcus section as a whole may thus be replaced with another sulcus section of different geometry. The cooperating teeth 50 and 53 act as the locking mechanism which allows for adjustability of the distance between the sulcus bar 40 and the orthotic body 12, as well as locking of the selected distance.

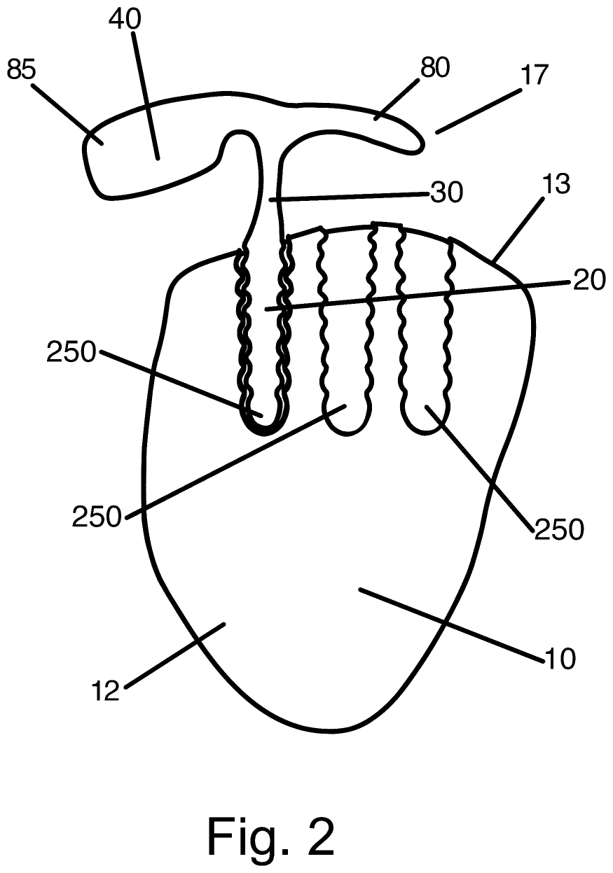

FIG. 2 depicts schematically a simplified top view of an aspect of the invention allowing lateral adjustability of the sulcus portion 17 relative to body section 12. The body section has a plurality of laterally arranged cavities 250 and the stem 20 may be located in any of the cavities of this plurality of cavities. Similar to other embodiments, the cavities are individually dimensioned to cooperate with the stem 20. The stem 20 and the cavities 250 provide a matching and complimentary arrangement of indentations and protrusions similar to the teeth of FIG. 1, however the protrusions and indentations are depicted as undulations. Different spacing between the laterally arranged cavities 250 may be utilized, such as, by way of example 2 mm or 3 mm, or 4 mm or 5 mm or 6 mm or 7 mm, and even up to 10 mm. The spacing may equal the cavity width or differ therefrom. Various types of protrusions and indentations may be utilized in the varying embodiments disclosed herein, and the type of such protrusions and indentations is a matter of technical choice. Notably, the foot orthotic body depicted in FIG. 2 extends only partially under the foot, which demonstrate that the orthotic body may be partial in any of the depicted embodiments, and the term body, as related to an orthotic may take varied shapes of the frontal region and the rest of the orthotic. As stated above, the foot orthotic does not need to extend the full length of the foot and by way of example, even a metatarsal pad may act as the foot orthotic.

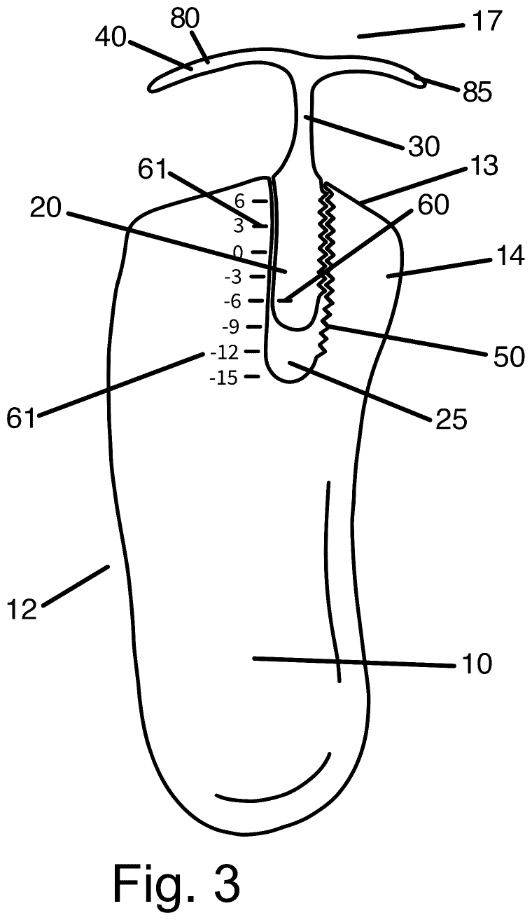

FIG. 3 depicts a top view of an embodiment where the cavity 25 is cut through the entire thickness of the orthotic body 12, at the forefoot portion 14 thereof. While in the embodiment of FIG. 1 stem teeth 53 are disposed on both side of the stem, in the embodiment depicted in FIG. 3 the teeth are disposed only on one side of the stem. This is yet another implementation detail shown to demonstrate various locking mechanisms which may be used in any of the embodiments. FIG. 3 also depicts another optional feature, where hatch mark 60 cooperates with a scale 61 and provides a dimensional reference of the front to back displacement of the sulcus section 17 relative to the orthotic body 12. Clearly, the location of the scale 61 and the hatch mark 60 may be reversed between the orthotic body and the stem.

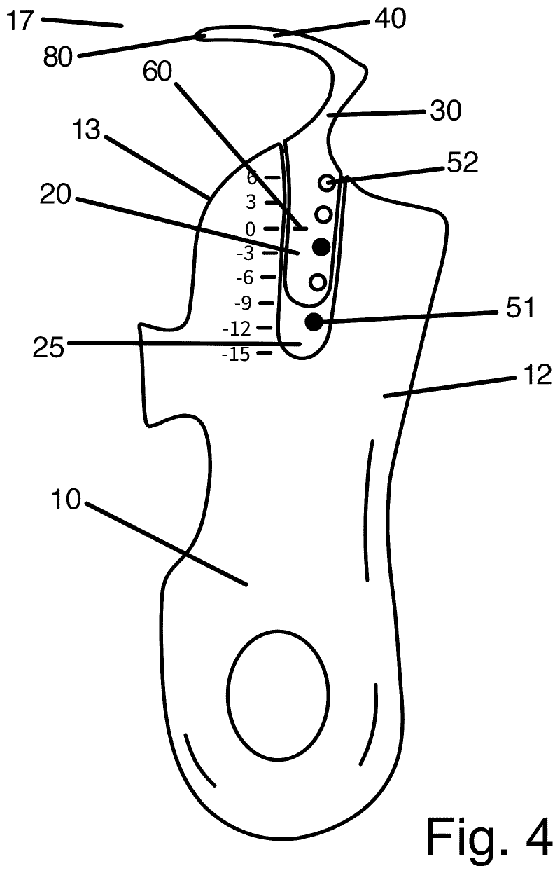

FIG. 4 depicts a top view of a left foot orthotic. In this embodiment the cavity 25 is formed in the top surface of the body, and the stem is engaged from the top. This embodiment depicts yet another optional engagement/locking arrangement comprising one or more pegs 51 attached to the body of the orthotic, and holes 52 at intervals in the stem 20. The pegs act as the protrusions and the holes as the indentations, and their interaction provides the adjustability and the lockability. The shape of the sulcus bar in the embodiment depicted in FIG. 4 lacks the medial pad portion 85 of the sulcus bar to demonstrate that the shape and design of the sulcus bar 40 and the sulcus section 17 as a whole may be modified for enhanced comfort.

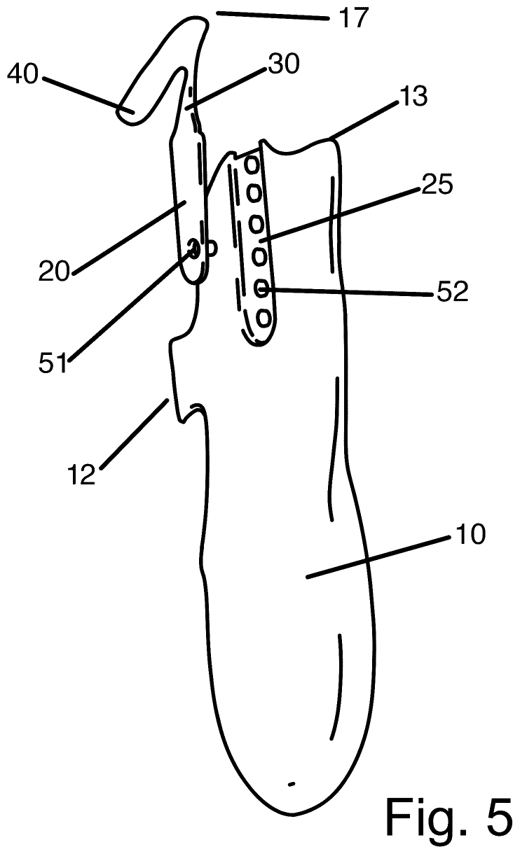

FIG. 5 depicts a bottom oblique view of another embodiment utilizing one or more peg shaped protrusions 51 (one shown) and a plurality of matching holes 52 forming the cooperating indentations. The sulcus section 17 is shown disengaged from the body section 12. Pegs 51 are disposed on the stem 20 and matching holes 52 are formed in the body of the orthotic. It is noted that the holes 52 may or may not penetrate all the way through the body section 12.

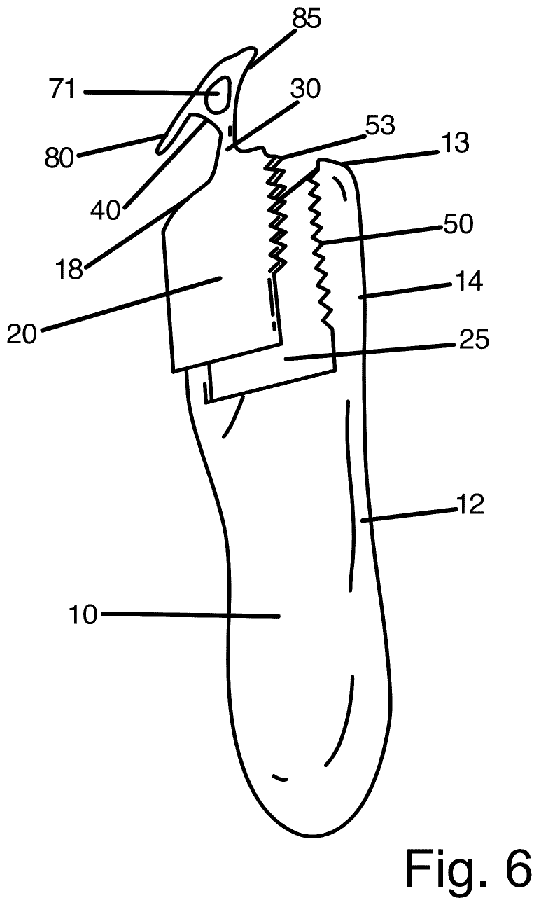

FIG. 6 depicts a right orthotic bottom oblique view of an embodiment having an alternative shaped sulcus bar 40 which defines a hole 71 designed to surround the second metatarsal head. The hole 71 demonstrates one more optional feature which may be incorporated into any of the sulcus device embodiments depicted herein and is provided to demonstrate the high variability of sulcus device designs for enhancing comfort. In the embodiment of FIG. 5 the stem 20 is wide and includes the leading edge 18 of part of the forefoot portion 14 of the orthotic. Such arrangement may offer several advantages. In one arrangement the stem 20 forms the leading edge 18 of the forefoot portion of the body of the orthotic, and thus the leading edge 18 of the stem allows adjustability of the effective length and position from the leading edge of the orthotic frontal region 13.

In certain embodiments, a plurality of sulcus sections 17 are provided, and such sulcus sections may be formed to provide differing fore-to-aft and/or lateral adjustments of the distance between the sulcus bar and the orthotic, as well as adjustments to the shape and location of the orthotic body effective leading edge, formed by the stem leading edge 18.

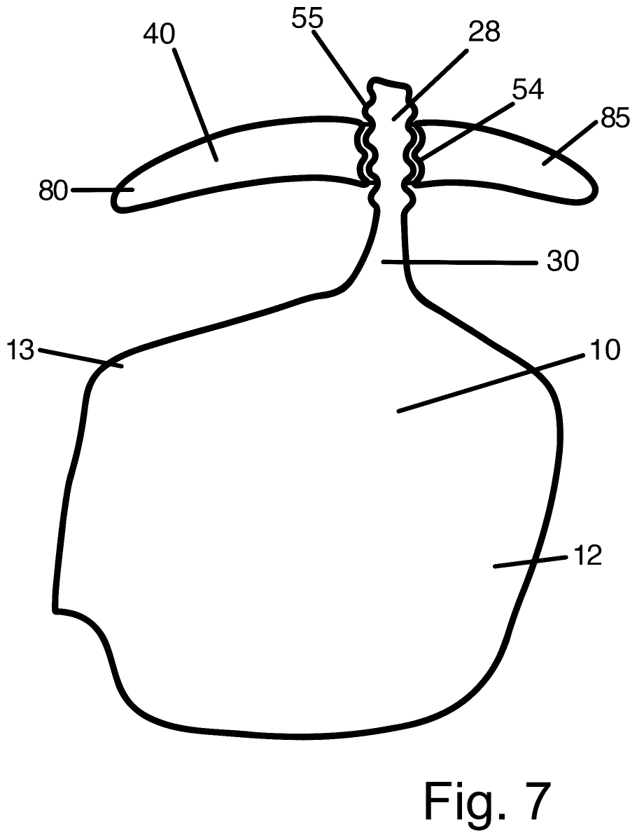

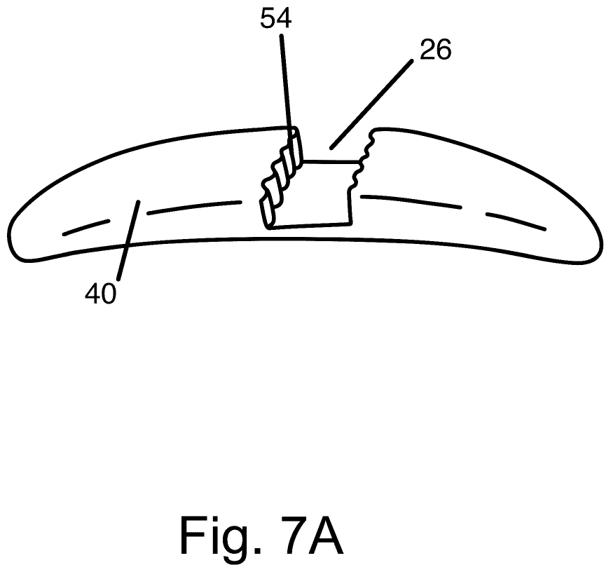

FIG. 7 depicts an alternative embodiment where the intermetatarsal bar 30 and the sulcus bar 40 form the cooperating parts which allow the for-aft adjustment of the sulcus bar relative to the body section 12. In this embodiment, the intermetatarsal bar 30 forms a portion of the body section 12 rather than the sulcus section 17, and a forward portion of the intermetatarsal bar also forms the stem, which is constructed to cooperate with a cavity 26 in the sulcus bar 40 rather than with a cavity 25 in the orthotic body, which in this embodiment is superfluous. The locking mechanism comprises protrusions and indentations, which are depicted in this embodiment in the distal portion 28 of the intermetatarsal bar 30 as cooperating and matching undulations 55, disposed on the intermetatarsal bar 30 and undulations 54 disposed on the sulcus bar 40. In this example the body of the orthotic 10 is primarily under the forefoot.

FIG. 7A depicts an oblique view of a sulcus bar 40 of FIG. 6. The cavity 26 is located in the sulcus bar. The undulations 54 are formed in the cavity side wall.

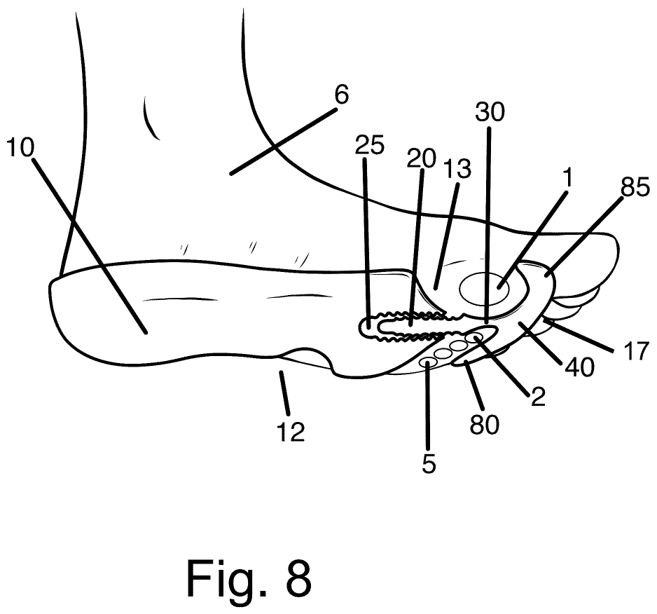

FIG. 8 depicts schematically a foot 6 operationally positioned on top of an embodiment of a sulcus device embodied as an orthotic 10 which utilizes an adjustable sulcus bar. In the depicted embodiment, metatarsal heads 1, 2 and 5 are located behind the sulcus bar 40 and a portion the stem 20 and the intermetatarsal bar 30 are disposed between the first metatarsal head 1 and second metatarsal head 2, however the stem and/or the intermetatarsal bar may be located at any desired location along the frontal region 13.

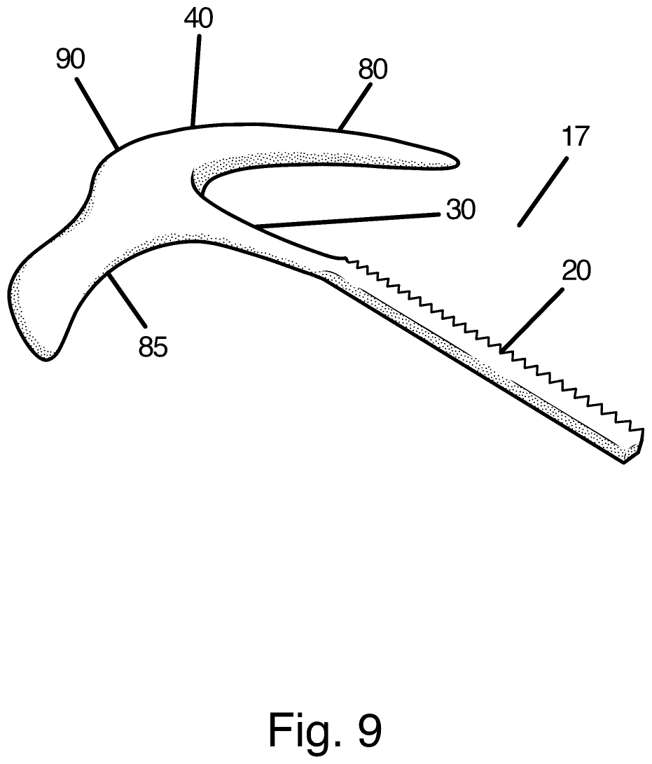

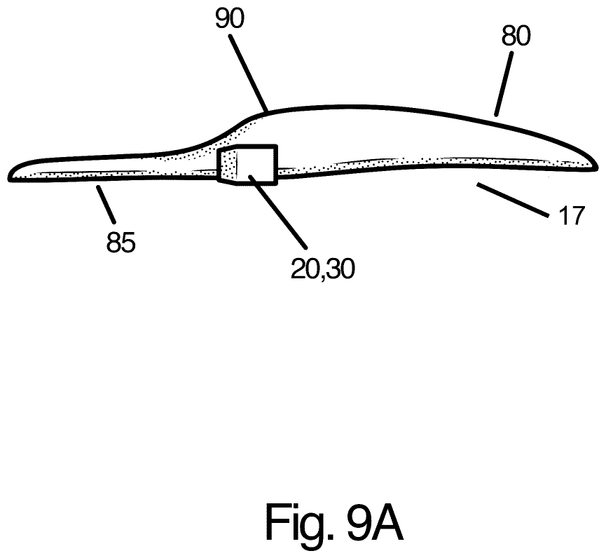

FIG. 9 depicts an isometric view of a sulcus device embodied as a discrete sulcus section 17, which utilizes a stem 20 and a intermetatarsal bar 30 as in some other embodiments, however this embodiment does not require a shoe or an orthotic, but instead is formed to cooperate with a matching shoe or orthotic. The sulcus bar 40 is shown to have the lateral pad portion 80 further having a lateral height zone 90, which has a greater height than other portions of the pad. The lateral height zone 90 is disposed about the IM bar and extending laterally therefrom, such that operationally it lies in the sulcus cavity area forward and between the first and second metatarsal heads, and or forward of at least the second metatarsal head. Therefore, a portion of the lateral height zone may extend over the IM bar, or begin adjacent thereto. Commonly but not mandatorily, the thickest area of the lateral height zone is disposed about the intermetatarsal bar region and the height tappers as the lateral pad extends laterally. The lateral pad portion 80 at least at this embodiment, arcs rearwardly. This may be seen in FIG. 9A, which is a rear side view of the embodiment in FIG. 9. Optionally, the lateral pad portion 80 is extended sufficiently to lie under the plantar sulcus cavity about the fifth metatarsal, as depicted by way of example in FIG. 7, and in some embodiments the lateral pad portion extends only sufficiently to lie in the sulcus cavity near the fourth, third or, in cases of a short lateral pad, the second metatarsal head.

A lateral height zone 90 and a rearwardly arcing lateral pad 80 may be added to any and all disclosed embodiments of the sulcus device.

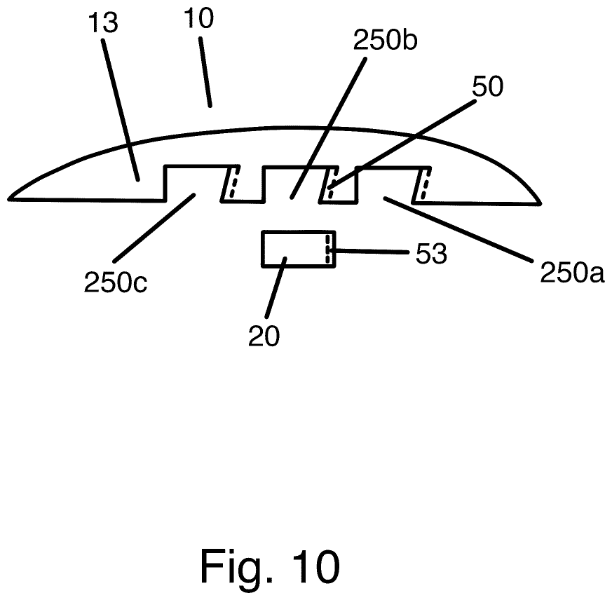

FIG. 10 depicts a rearwards looking view of the frontal region 13 of an orthotic 10 embodiment utilizing a plurality of cavities 250a-c to offer lateral adjustment of the sulcus section 17. As seen, the cavities do not conform precisely to the stem 20. This construction is advantageous for maintaining the stem in place during placement of the orthotic into the shoe. In the depicted embodiment the stem or a portion thereof is more deformable than the orthotic body. During insertion of the stem into the desired cavity the stem is compressed to deform sufficiently to allow insertion and once inserted in the cavity, the portion of the stem engaging the cavity expands to at least partially conform to the slanted edge of the selected cavity 250. While teeth 50, 53 on the cavity and the stem respectively engage, the stem is held in place by the elastic force to facilitate the placement of the orthotic and the sulcus section into the shoe. The stem depicted is similar to the stem shown in FIG. 1, however this association is not mandatory and the slanted slot wall may be utilized with numerous other sliding and/or locking arrangements. It is noted that the deformable stem described herein may be utilized in any of the embodiments using a stem, and such construction as shown in FIG. 10 may be utilized in a single cavity 25 embodiments as well as in a plurality of substantially parallel cavities 250, and that the geometrical structure of the cavities do not have to all be identical.

Notably, the cavity 25 or any of the cavities 250 in body section 12 has a matching shape to accept and guide the placement of the stem within the cavity, however this is not mandatory and the cavity need only be dimensioned to receive the stem, or a part thereof, within the cavity, in either compressed or natural form. In the depicted example the cross section is trapezoidal but other shapes such as triangular, keyed shape, and the like, are explicitly considered.

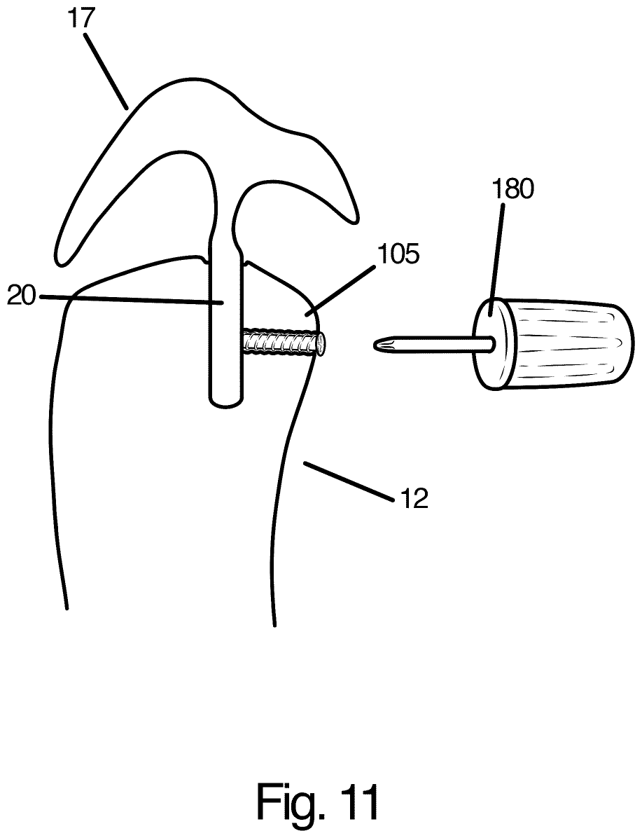

FIG. 11 depicts a simplified top view cross section of an embodiment showing an optional locking mechanism comprising a screw 105. A threaded cavity is formed in the orthotic body 12 such that a screw 105 can be advanced so as to pinch the stem 20 at a desired location within the body cavity where the screw locks the stem 20, and thereby the sulcus section 17, in a desired distance between the sulcus bar and the orthotic body. A screwdriver 180 is shown adjacent to the screw for illustration purposes, and is not a part of the invention.

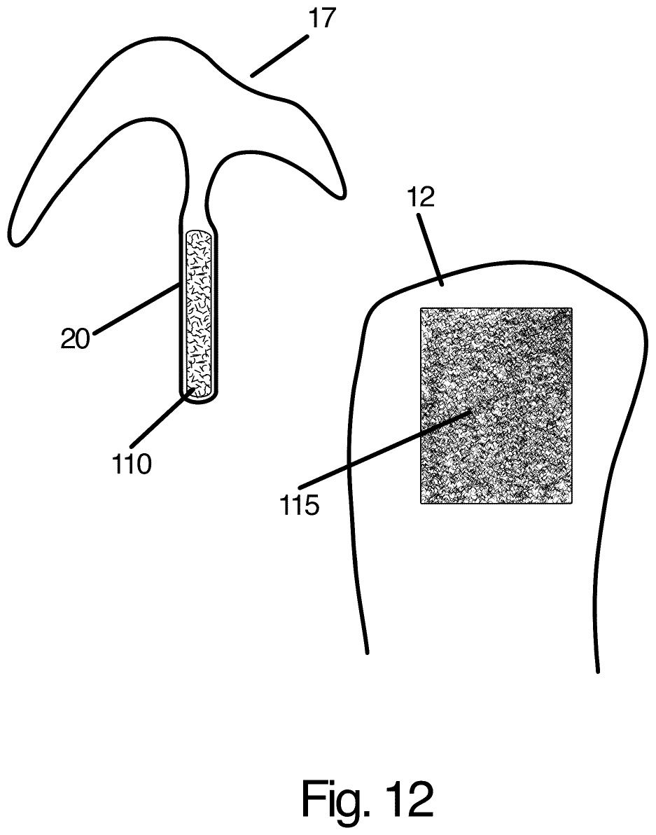

FIG. 12 depicts yet another embodiment of an adjustment and locking mechanism, comprising two cooperating hook and loop parts which together form a fastening. One part 110 of the cooperating hook and loop fastening is coupled to the stem 20 and the other cooperating hook and loop part 115 is coupled to the orthotic body 12 or to the shoe inner sole. This arrangement allows wide and precise adjustment of the lateral and longitudinal position of the sulcus portion by positioning the stem hook and loop part 110 at any desired location on the orthotic body part 115. A very flat stem is desired but not mandatory for such embodiments. It is noted that such embodiment of the locking mechanism may be utilized in an aspect of the sulcus device which is embodied in footwear by placing one hook and loop part on the inner sole of the footwear, and in an orthotic by placing the hook and loop part either on the top or on the bottom face of the orthotic.

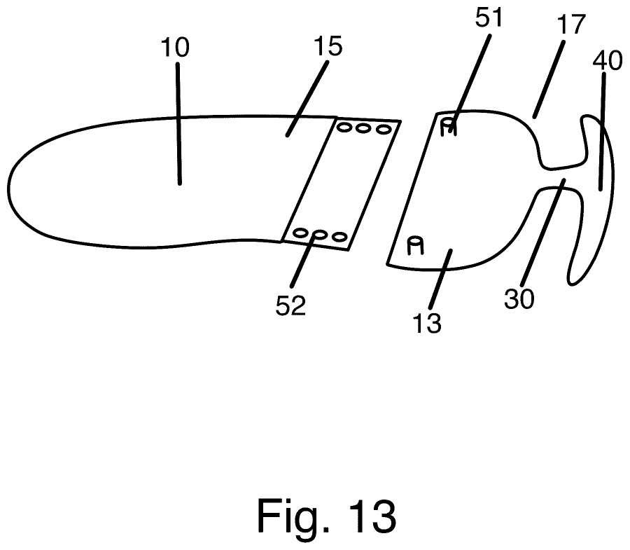

FIG. 13 depicts an embodiment where the orthotic 10 rear region 15 and its frontal region 13 are separated and are joinable by a peg 51 and hole 52 arrangement. The frontal region 13, which includes the sulcus section 17, is adjustably attached by cooperating pegs 51 and matching plurality of holes 52 in the rear region. Placing the pegs in varying holes changes the for and aft distance of the sulcus region from the rear of the foot and the distance between the leading edge of the orthotic is fixed, however this embodiment allows higher rigidity of the orthotic. The front portion with the sulcus portion may be replaced to provide individualize distances, sizes and shapes of the sulcus bar and/or sulcus section.

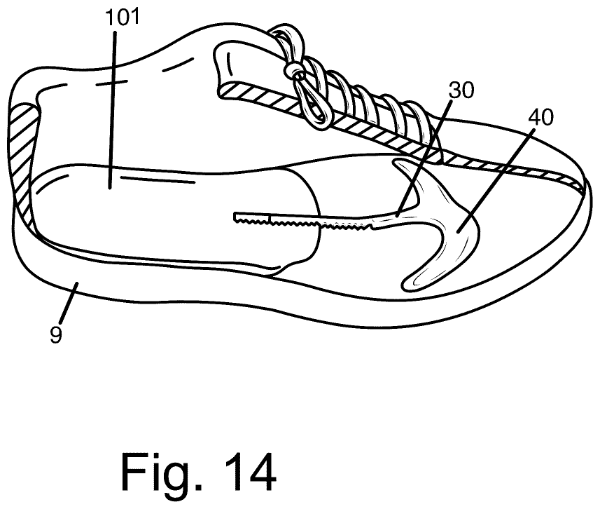

FIG. 14 depicts yet another embodiment of the sulcus device, where the sulcus device is adjustably embedded within the structure of the shoe. The sulcus device 17 of FIG. 14 is in the form of the IM bar 30 and the sulcus bar 40. As seen, the sulcus device is similar to the sulcus device depicted in other figures such as FIG. 9, however the stem is adjustably received in a cavity in the inner sole 101, which forms on part of the locking mechanism. While the locking mechanism depicted shows an interacting teeth based mechanism, any other mechanism may be shown. While not shown, a plurality of parallel slots may be utilized as show by way of example in FIGS. 2 and 10.

The term inner sole relates to the upper side of the shoe sole, which resides inside the shoe, however such inner sole may not be the topmost layer of the sole as it, or portions thereof, may be covered by one or more pliable layers such as foam, cloth and the like.

Various figures depict varying optional shapes of the orthotic body 12. These differences are shown to demonstrate that varying forms of the orthotic body may be utilized and such differences do not materially change the nature of the embodiment as long as the body which is utilized for the orthotic 10 is capable of receiving and/or supporting a sulcus section providing side-to-side or longitudinal position adjustment and locking. Stated differently, any of the sulcus sections depicted and disclosed herein may be attached to any orthotic body type, including a partial orthotic body such as a metatarsal pad by way of example, as long as such body allows the coupling of the sulcus section in a manner that allows lateral and/or longitudinal adjustment and subsequently locking the sulcus section at the adjusted disposition.

In some embodiments, the sulcus device is a discrete sulcus section component configured for cooperating with a matching structure in an orthotic body or in footwear, which may be separately provided. Each of the sulcus sections depicted and/or disclosed herein, and their equivalents, may be provided as a discrete component constituting a sulcus device, and the claims extend to such embodiments.

In certain implementations, the orthotic is provided with a plurality of discrete sulcus sections or portions of the sulcus sections for a single orthotic, and individual users may select the desired sulcus section that fits their needs.

It is noted that any of the embodiments may utilize any desired set of protrusions and indentations for enacting a locking mechanism, separately or in combination, and that various body embodiments may be combined with any compatible sulcus section. Similarly, other locking mechanism, such as the hook and loop locking mechanism, the screw based locking mechanism, and the like.

For brevity, this disclosure discusses certain anatomical bony structure such as metatarsal heads, phalanx, and the like, however the skilled in the art would readily understand that the bony structures themselves are not in direct contact with the orthotic, the sulcus bar, and the like, but instead a skin layer, and commonly a fat pad underlies the bony structure and the skin or fat pad is the body portion which comes in contact, or is surrounded by, or is disposed in proximity to, the various portions of the orthotic and/or the sulcus bar. It is further noted that the term “operational” and its various forms means that the description relates to the respective element being in normal use, underlying the user's foot. The term “underfoot structure” refers to any and all sections of the sulcus device differing from a sulcus bar, an intermetatarsal bar, a stem (if utilized), and the like. The term “bar” relates generally to an elongated and generally narrow structure rising upwardly, but does not mandate a straight elongated shape. As may be seen in the drawings, must sulcus bars are curved. The term height implies an elevation towards the foot bottom. Unless otherwise specified, relational terms used in this disclosure should be construed to include certain tolerances that the skilled in the art would recognize as providing equivalent functionality. Adjectives such as “about”, “adjacent”, and “substantially” that modify a condition or relationship characteristic of a feature or features of an embodiment of the present technology, are to be understood to mean that the condition or characteristic is defined to within tolerances that are acceptable for operation of the embodiment for an application for which it is intended, or sufficiently close to the location, disposition, or configuration of the relevant element to preserve operability of the element within the invention which does not materially modifies the invention. Similarly, unless specifically specified or clear from its context, numerical values should be construed to include certain tolerances that the skilled in the art would recognize as having negligible importance as it does not materially change the operability of the invention. When the term “about” precedes a numerical value, it is intended to indicate +/−15%, or +/−10%, or even only +/−5%, and in some instances the precise value. It is further noted that whenever a sulcus section or portions thereof are said to be placed in specific posture, such placement is done within acceptable tolerances, as precise disposition is not required and commonly only an approximation is practical. Thus, by way of example, when a stem is disposed in one of a plurality of cavities to provide lateral adjustment, the most comfortable cavity may be selected, while it is possible to that an intermediate location between the cavities may provide more precise placement, but such disposition is not provided by a specific embodiment.

Notably, whenever the term ‘and/or’ is used in this disclosure and the attached claims, it should be construed as any number, combination or permutation of all, one, some, a plurality or none of each of the item or list mentioned. It is also understood that “(s)” appended to the end of a word designates either singular or plural of the word. It is further understood that “or” is an inclusive “or” to include all items in a list and not intended to be limiting and means any number, combination or permutation of all, one or plurality of each of the item or list mentioned, unless the term ‘or’ is explicitly defined as exclusive, or if the context would clearly indicate an exclusive or to the skilled artisan. In the description and claims of the present disclosure, each of the verbs, “comprise” “include” and “have”, and conjugates thereof, are used to indicate that the object or objects of the verb are not necessarily a complete listing of features, members, components, elements, steps or parts of the subject or subjects of the verb.

In this disclosure reference is often made to the accompanying drawings which form a part of the disclosure, and in which are shown by way of illustration and not of limitation, exemplary implementations and embodiments. Further, it should be noted that while the description provides various exemplary embodiments, as described and as illustrated in the drawings, this disclosure is not limited to the implementations described and illustrated herein, but can extend to other embodiments as would be known or as would become known to those skilled in the art. Reference in the disclosure to “one embodiment”, “this embodiment”, “these embodiments”, “several embodiments”, “selected embodiments”, “some embodiments” or conjugates thereof means that a particular feature, structure, or characteristic described in connection with the relevant embodiment(s) may be included in one or more implementations and/or embodiments, and the appearances of these phrases in various places in the specification are not necessarily all referring to the same embodiment(s). Additionally, in the description, numerous specific details are set forth in order to provide a thorough disclosure, guidance and/or to facilitate understanding of the invention or features thereof. However, it will be apparent to one of ordinary skill in the art that these specific details may not all be needed in each implementation. In certain embodiments, well-known structures and materials have not been described in detail, and/or may be illustrated schematically or in block diagram form, so as to not unnecessarily obscure the disclosure. Stated differently, features described as applying to one embodiment may be incorporated with other embodiment(s), and all described features are required for any or all of the disclosed embodiments. In some cases, a single numeral may be utilized to describe both a plurality of items and any individual item therefrom. By way of example, a plurality of cavities and each and every cavity therefrom are enumerated 250, while a single cavity may be enumerated 25.

For clarity the directional terms such as ‘medial’, ‘lateral’, ‘anterior’, ‘posterior’, ‘proximal’, ‘distal’, ‘inferior’, ‘superior’, ‘up’, ‘down’, ‘left’, ‘right’, and descriptive terms such as ‘upper’ and ‘lower’, ‘above’, ‘below’, ‘sideways’, ‘inward’, ‘outward’, and the like, are applied according to their ordinary and customary meaning, to describe relative disposition, locations, and orientations of various components. When relating to the drawings, such directional and descriptive terms and words relate to the drawings to which reference is made. Notably, the relative positions are descriptive and relative to the above described orientation such as an orientation which would be exercised during upright walking and/or standing and modifying the orientation would not change the disclosed relative structure. Positional or motional terms such as “upper”, “lower”, “right”, “left”, “bottom”, “below”, “lowered”, “low”, “top”, “above”, “elevated”, “high”, “vertical”, “horizontal”, “front”, “back”, “backward”, “forward”, “upstream” and “downstream”, as well as grammatical variations thereof, may be used herein for exemplary purposes only, to illustrate the relative positioning, placement or displacement of certain components, to indicate a first and a second component in present illustrations or to do both. Such terms do not necessarily indicate that, for example, a “bottom” component is below a “top” component, as such directions, components or both may be flipped, rotated, moved in space, placed in a diagonal orientation or position, placed horizontally or vertically, or similarly modified. The terms ‘lateral’, “medial”, and the like are given their common anatomical meanings, unless otherwise specified or is clear from the context.

Although the foregoing invention has been described in detail by way of illustration and example, it will be understood that the present invention is not limited to the particular embodiments, options, alternatives, and examples provided in the description and the drawings. Specific embodiments described but may comprise any combination of the above disclosed elements and their equivalents and variations thereof, as well as those combinations, changes and/or modifications which will be obvious to those skilled in the art in view of the present disclosure. The invention extends to such variations and modifications as fall within the true spirit and scope of the invention.

1. A foot orthotic comprising:
a body section having a frontal region;
a sulcus section at least partially disposed forwardly to the frontal region, the sulcus section comprising an elongated intermetatarsal bar having a longitudinal axis, and a sulcus support bar coupled thereto;
the sulcus support bar comprising at least one pad disposed forwardly of the frontal region and at an adjustable distance therefrom, the distance being adjustable at least in a front to rear direction;
the elongated intermetatarsal bar coupling between to the body section and the sulcus support bar; and,
a locking mechanism adapted to selectively lock the sulcus support bar and the body section at the adjustable distance therebetween.

2. An orthotic as claimed in claim 1, wherein the intermetatarsal bar may be formed integrally with, or be coupled to, the sulcus support bar.

3. An orthotic as claimed in claim 1, wherein the sulcus section further comprises a rearwardly extending stem, at least a portion of the stem is dimensioned to be receivable within an elongated cavity defined by the body section of the orthotic, be adjustably located therein and selectively locked at a distance relative thereto by the locking mechanism.

4. An orthotic as claimed in claim 3, wherein the locking mechanism comprises a screw locking the stem relative to a portion of the body section.

5. An orthotic as claimed in claim 3, wherein the body section defines a plurality of substantially parallel elongated cavities, each cavity being dimensioned to receive the stem or a portion thereof therein, the cavities being parallelly disposed along a lateral axis of the orthotic.

6. An orthotic as claimed in claim 1, wherein the locking mechanism comprises a hook and loop fastening.

7. An orthotic as claimed in claim 1, wherein the pad comprises a lateral portion forming a backwardly extending arch.

8. A foot orthotic as claimed in claim 1, wherein the pad further comprised a medial portion disposed on an opposing side of the intermetatarsal bar longitudinal axis from the lateral portion, the lateral portion of the pad or a portion thereof being higher than an average height of the medial portion of the pad.

9. A foot orthotic as claimed in claim 1 wherein the elongated intermetatarsal bar is formed integrally with the body and extending forwardly therefrom, and wherein the sulcus bar being adjustably coupled and lockable to the intermetatarsal bar.

10. A sulcus section adapted for use in a foot orthotic having a body section, or in a shoe having an inner sole, the inner sole or the orthotic having an elongated cavity formed therein, the cavity having a front end, the shoe or orthotic having a first part of a locking mechanism, the sulcus section comprising:
an elongated intermetatarsal bar having a longitudinal axis;
a stem coupled to or integral with the intermetatarsal bar, the stem being dimensioned to be at least partially received within the cavity;
a sulcus support bar formed integrally to, or coupled to, the intermetatarsal bar;
the stem forming a second part of the locking mechanism, which when cooperating with the first part of the locking mechanism, locks the stem at an adjustable position thereby locking the distance between the sulcus bar and the front end of the cavity.

11. A sulcus support section as claimed in claim 10, wherein the sulcus support bar comprises a medial pad extending medially to the longitudinal axis of the intermetatarsal bar axis.

12. A sulcus section as claimed in claim 10, wherein the sulcus support bar comprises a lateral pad portion disposed on a lateral side of the intermetatarsal bar axis.

13. A sulcus section as claimed in claim 12, wherein the lateral pad portion of the pad or a portion thereof forming a backwardly extending arch.

14. A sulcus section as claimed in claim 12, wherein the sulcus support bar further comprises a medial pad extending medially to the longitudinal axis of the intermetatarsal bar axis

15. A sulcus section as claimed in claim 14, wherein the lateral pad portion further comprises a lateral height zone having a greater height than an average height of the medial pad portion.

16. A sulcus section as claimed in claim 10, wherein the stem comprises at least a portion of the intermetatarsal bar, and having a plurality of teeth formed in at least one side thereof.

17. A sulcus bar as claimed in claim 16, wherein the teeth are dimensioned to engage at least one protrusion in the cavity of the body.

18. A shoe having an inner sole and constructed to cooperate with a sulcus section as claimed in claim 10, the inner sole having at least one elongated cavity formed therein the cavity being dimensioned to receive at least portion of the stem therein, the inner sole incorporating the first portion of a locking mechanism.

19. A shoe as claimed in claim 18, wherein the inner sole comprises a plurality of elongated cavities, each dimensioned to receive at least portion of the stem therein, the cavities being substantially parallel to each other and extending along a lateral axis of the shoe.
